(19) United States
(12) Patent Application Publication
(10) Pub. No.: US 2004/0260181 A1
(43) Pub. Date: Dec. 23, 2004

(54) Repolarization system which repolarizes transducer used in ultrasonic probe

(75) Inventors: Yasuhisa Makita, Tochigi-ken (JP); Hiroyuki Shikata, Tochigi-ken (JP); Katsutoshi Yuasa, Tochigi-ken (JP); Takashi Ogawa, Tochigi-ken (JP)

Correspondence Address: OBLON, SPIVAK, MCCLELLAND, MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-Shi (JP)

(21) Appl. No.: 10/768,006

(22) Filed: Feb. 2, 2004

(30) Foreign Application Priority Data
Jan. 31, 2003 (JP) 2003-024554

(51) Int. Cl. A61B 8/14

(52) U.S. Cl. 600/459

(57) ABSTRACT

A repolarization system including an ultrasonic probe includes a plurality of transducers, a voltage generator, a switch, a connector, an interface, and a controller. The plurality of transducers are provided in the ultrasonic probe. Each one or more of the transducers is provided with a pair of electrodes. The voltage generator is configured to generate a predetermined voltage high enough to repolarize the transducers. The switch is configured to switch to supply the voltage to the pair of electrodes. The connector is configured to connect the pair of electrodes to the switch through a plurality of lands. Each of the plurality of lands corresponds to the pair of electrodes. The lands are provided to keep insulation enough to repolarize the transducers among the lands. The interface is configured to give an instruction. The controller is configured to control the voltage generator to generate the predetermined voltage. The controller is further configured to control the switch to supply the predetermined voltage to the pair of electrodes in accordance with the instruction.

### CROSS-REFERENCE TO RELATED APPLICATION

This application is based upon and claims the benefit of priority from prior Japanese Patent Application No. P2003-24554, filed on Jan. 31, 2003, the entire contents of which are incorporated herein by reference.

### BACKGROUND OF THE INVENTION

1. Field of the Invention

The present invention relates to a repolarization system which repolarizes transducers used in an ultrasonic probe for generating an ultrasound. The present invention further relates to the ultrasonic probe and to an ultrasound imaging apparatus using the repolarization system.

2. Discussion of the Background

As known in the field of an ultrasound imaging apparatus, an ultrasonic probe used as a part of the ultrasound imaging apparatus includes a plurality of transducers. The transducers are supplied with a high voltage and accordingly generate an ultrasound. The generated ultrasound is insonified towards an object or a patient. The insonified ultrasound returns from the object or the patient as an echo signal. The echo signal is converted into an electronic signal so as to obtain an ultrasound image. The obtained ultrasound image is displayed for observation. Such an ultrasound imaging apparatus is used, for example, as an ultrasound flaw detector (or a reflectoscope) for detecting flaws caused inside a welded part of metals. Further, such an ultrasound imaging apparatus is also used as an ultrasound diagnosis apparatus for diagnosing the inside of a patient. The metal, the patient, or any other object to be insonified by the ultrasound imaging apparatus is hereinafter referred to as a specimen.

An ultrasonic probe used for an ultrasound diagnosis apparatus typically includes tens to hundreds of transducers arranged in an array. The number of the transducers tends to increase as a higher resolution is required for images.

Each one or more of transducers are provided with a pair of electrodes in the ultrasound imaging apparatus. When the pair of electrodes is supplied (or impressed) with a predetermined high voltage, the transducer generates an ultrasound. A polarization characteristic of the electrodes, however, usually deteriorates during a manufacture of the transducer and/or after a use for a predetermined time. As a result of the deterioration, an acoustic characteristic deteriorates in the ultrasonic probe. Accordingly, image quality deterioration is caused in a displayed ultrasound image.

In order to solve the above issue, various techniques have been introduced to restrain a depolarization in the transducer (i.e., the deterioration of a polarization characteristic in a transducer). For example, Japanese Patent Application Publication No. PH7-99348 describes a piezoelectric monocrystal, an ultrasonic probe, and an array-type ultrasonic probe. Each of those can be restrained the depolarization during the manufacture and acoustic characteristic deterioration due to its long time use. According to the description, the depolarization is restrained by giving a specific condition to a monocrystal included in a transducer.

Another exemplary technique is described in Japanese Patent Application Publication No. PH10-93154. According to the description, it is possible to obtain a transducer having a high electromechanical coupling factor (or an acoustic characteristic) by restraining the depolarization of the transducer during a manufacture of the transducer. The restraint is accomplished by preparing the transducer with a Perovskite-type lead compound oxide monocrystal and giving a specific condition to a cutting surface shape of the transducer.

The techniques described above can be useful to restrain the depolarization of transducers and accordingly to postpone a time of the depolarization. Once, however, the transducers are so depolarized that a preferable image quality cannot be kept any more, the techniques cannot cope with this problem. Since there is no technique of appropriately solving this problem, an ultrasonic probe including the transducers is required to be replaced with a new one.

Meanwhile, it is generally known that a depolarized transducer can be repolarized by supplying (or impressing) a predetermined high voltage to its pair of electrodes. That is, it is possible to recover an acoustic characteristic of the transducer. A conventional ultrasonic probe is, however, not configured from a point of view of reusing its transducers by repolarization. Therefore, it is not possible to directly supply a predetermined high voltage to transducers due to the configuration of the conventional ultrasonic probe. In order to supply the predetermined high voltage for the repolarization, it is necessary to disassemble the ultrasonic probe, pick up the transducers, and then supply the predetermined high voltage to the transducers (or to the pair of electrodes of each transducer). Such a configuration will be compared to an embodiment of the present invention in ‘DESCRIPTION OF THE PREFERRED EMBODIMENTS’ of the present invention.

Since the above repolarization requires a lot of work and even reassembling the ultrasonic probe after the repolarization, it is not implemented in practice. In addition, a conventional ultrasound imaging apparatus has no means of informing of (or giving a notice of) when the depolarization will be estimated to occur. Therefore, an operator has to determine a replacement time in accordance with image quality deterioration based on displayed images.

### SUMMARY OF THE INVENTION

According to a first aspect of the present invention, there is provided a repolarization system including an ultrasonic probe. The system includes a plurality of transducers, a voltage generator, a switch, a connector, an interface, and a controller. The plurality of transducers are provided in the ultrasonic probe. Each one or more of the transducers is provided with a pair of electrodes. The voltage generator is configured to generate a predetermined voltage high enough to repolarize the transducers. The switch is configured to switch to supply the voltage to the pair of electrodes. The connector is configured to connect the pair of electrodes to the switch through a plurality of lands. Each of the plurality of lands corresponds to the pair of electrodes. The lands are provided to keep insulation enough to repolarize the transducers among the lands. The interface is configured to give an instruction. The controller is configured to control the voltage generator to generate the predetermined voltage. The controller is further configured to control the switch to supply the predetermined voltage to the pair of electrodes in accordance with the instruction.

According to a second aspect of the present invention, there is provided an ultrasound imaging apparatus for obtaining an ultrasound image through an ultrasonic probe. The apparatus includes a plurality of transducers, a voltage generator, a switch, a connector, an interface, and a controller. The plurality of transducers are provided in the ultrasonic probe. The plurality of transducers are configured to generate an ultrasound so as to obtain the ultrasound image. Each one or more of the transducers is provided with a pair of electrodes. The voltage generator is configured to generate a predetermined voltage high enough to repolarize the transducers. The switch is configured to switch to supply the voltage to the pair of electrodes. The connector is configured to connect the pair of electrodes to the switch through a plurality of lands. Each of the plurality of lands corresponds to the pair of electrodes. The lands are provided to keep insulation enough to repolarize the transducers among the lands. The interface is configured to give an instruction. The controller is configured to control the voltage generator to generate the predetermined voltage. The controller is further configured to control the switch to supply the predetermined voltage to the pair of electrodes in accordance with the instruction.

According to a third aspect of the present invention, there is provided an ultrasonic probe which is connectable to a voltage, supply apparatus. The probe includes a plurality of transducers, a plurality of lands, a cable, and a connector. Each one or more of the plurality of transducers is provided with a pair of electrodes. Each of the plurality of lands is configured to correspond to the pair of electrodes. The plurality of lands are provided to keep insulation enough to repolarize the transducers among the lands. The cable is configured to include a plurality of voltage supply lines connected to the lands, respectively. The connector is configured to connect the cable to the voltage supply apparatus. A predetermined voltage is supplied to the transducers through the lands.

### BRIEF DESCRIPTION OF THE DRAWINGS

A more complete appreciation of embodiments of the present invention and many of its attendant advantages will be readily obtained by reference to the following detailed description considered in connection with the accompanying drawings, in which:

FIG. 1 is an illustration showing an exemplary configuration of an ultrasonic probe according to embodiments of the present invention;

FIGS. 2A and 2B are illustrations showing a first exemplary configuration for explaining a land connection according to embodiments of the present invention;

FIG. 3 is an illustration showing a second exemplary configuration for explaining the land connection according to embodiments of the present invention;

FIG. 4 is an illustration showing an exemplary configuration for explaining a conventional land connection according to a prior art of the present invention;

FIG. 5 is a block diagram showing a first exemplary configuration of a repolarization system according to a first embodiment of the present invention;

FIG. 6 is a block diagram showing a second exemplary configuration of the repolarization system according to a second embodiment of the present invention;

FIG. 7 is a block diagram showing a third exemplary configuration of the repolarization system according to a third embodiment of the present invention;

FIG. 8 is a block diagram showing a fourth exemplary configuration of the repolarization system according to a, fourth embodiment of the present invention;

FIG. 9 is a block diagram showing a fifth exemplary configuration of the repolarization system according to a fifth embodiment of the present invention;

FIG. 10 is a block diagram showing a sixth exemplary configuration of the repolarization system according to a sixth embodiment of the present invention;

FIG. 11 is a block diagram showing a first exemplary configuration of an ultrasound imaging apparatus according to a seventh embodiment of the present invention;

FIG. 12 is a flowchart showing an example of operations of an ultrasound imaging apparatus according to an eighth embodiment of the present invention; and

FIG. 13 is a block diagram showing a second exemplary configuration of the ultrasound imaging apparatus according to a ninth embodiment of the present invention.

### DESCRIPTION OF THE PREFERRED EMBODIMENTS

Embodiments of the present invention will be described with reference to the accompanying drawings.

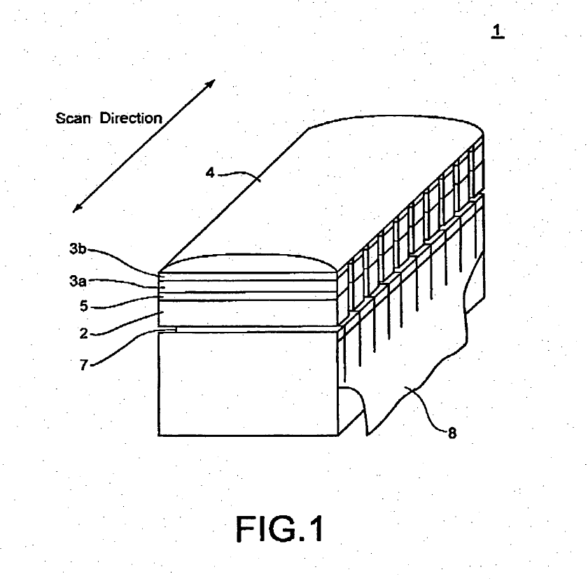

FIG. 1 is an illustration showing an exemplary configuration of an ultrasonic probe according to embodiments of the present invention. The ultrasonic probe can be used for an ultrasound imaging apparatus, such as, for example, an ultrasound flaw detector (or a reflectoscope) for detecting flaws caused inside a welded part of metals or an ultrasound diagnosis apparatus for the purpose of medical diagnoses.

As shown in FIG. 1, an ultrasonic probe 1 includes a plurality of transducers 2, acoustic matching layers 3a and 3b, an acoustic lens 4, electrodes 5 and 7, and a flexible printed wiring board 8. The transducers 2 are arranged in an array form and reversibly convert between ultrasound signals and electronic signals. The transducers 2 are aligned along a scan direction of ultrasound generated from the transducers 2. The acoustic matching layers 3a and 3b are formed on an ultrasound reception surface side of the ultrasonic probe 1. Although the acoustic matching layers 3a and 3b are provided as a bilayer configuration in FIG. 1, a single layer or more than two layers may be applicable as an acoustic matching layer configuration. The acoustic matching layers 3a and 3b are provided over the transducers 2 along the scan direction. The acoustic matching layers 3a and 3b are covered by the acoustic lens 4.

The electrodes 5 are provided to the transducers 2, respectively and are individual electrodes for supplying or impressing (hereinafter referred to as supplying) a predetermined high voltage to the transducers 2, respectively. Further, the electrodes 7 are also provided to the transducers 2, respectively and are individual electrodes for taking out electronic signals from the transducers 2, respectively. Each one of the transducers 2 is provided with one of the electrodes 5 and one of the electrodes 7. The one electrode 5 and the one electrode 7 can be in pairs. Alternatively, each adjacent two or more of the transducers 2 may be provided with one of the electrodes 5 land one of the electrodes 7. The one electrode 5 and the one electrode 7 can be in pairs. In this alternative case, the adjacent two or more transducers 2 commonly provided with the pair of the one electrode 5 and the one electrode 7 are operative in the same manner as if they constituted one transducer.

Lead wires from the electrodes 7 are connected to the flexible printed wiring board 8 by soldering or conductive paste, for example. Lead wires from the electrodes 5 are also connected to the flexible printed wiring board 8 by soldering or conductive paste, for example. Alternatively, the electrodes 5 may be connected to an earth board while the lead wires from the electrodes 7 are connected to the flexible printed wiring board 8. The earth board may be connected to the flexible printed wiring board 8.

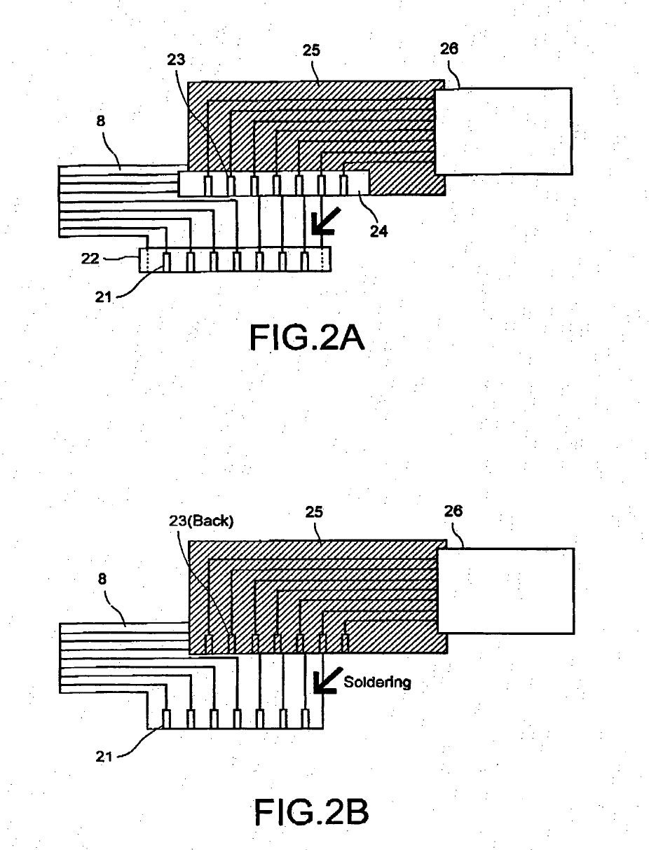

FIGS. 2A and 2B are illustrations showing a first exemplary configuration for explaining a land connection according to embodiments of the present invention. The land connection is implemented so as to connect the electrodes 5 and 7 (or transducers 2) and a cable of the ultrasonic probe 1.

As shown in FIG. 2A, the flexible printed wiring board 8 connected to the electrodes 5 and 7 has a connector 22. The connector 22 includes a plurality of lands 21. The lands 21 are connected to the electrodes 5 and 7 through voltage supply lines provided in the flexible printed wiring board 8. The lands 21 include signal lands and ground lands. Each of the signal lands is connected to each of the electrodes 7. Each of the ground lands is connected to one or more of the electrodes 5. In other words, one ground land can be used in common with a plurality of the electrodes 5.

In the connector 22, the lands 21 are provided to keep a predetermined interval between adjacent two of the lands 21 (i.e., between one of the lands 21 and a next of the one land 21). The predetermined interval is determined to be long enough to keep insulation between the adjacent two lands 21. This is because a predetermined high voltage is supplied to the lands 21 so that the acoustic characteristic of the transducers 2 is recovered. The predetermined high voltage may be six to seven times larger than a voltage to be supplied to the transducers 2 for ultrasound generation. Therefore, if a creepage distance for insulation is not kept as a land interval, a dielectric breakdown is caused in the land interval. Such a dielectric breakdown leads to a breakage of the ultrasonic probe 1.

The electrodes 5 and 7 are supplied with the predetermined high voltage through the flexible printed wiring board 8, the lands 21 (the connector 22), lands 23 (a connector 24), a printed wiring board 25, and a cable 26. The connector 22 is connected to the connector 24. When the connectors 22 and 24 are connected to each other, the lands 21 are connected to the lands 23. The lands 23 are prepared in a similar manner to the lands 21 so that each of the lands 21 is connected to corresponding one of the lands 23. Each of the lands 23 is also connected to each of voltage supply lines provided in the printed wiring board 25. The printed wiring board 25 is connected to the cable 26 which is also connected to an ultrasonic probe repolarization apparatus to be described later. The connection between the connector 22 and the connector 24 may be accomplished by soldering as shown in FIG. 2B so as to ensure the insulation between the adjacent two lands 21 and also between correspondingly adjacent two of the lands 23.

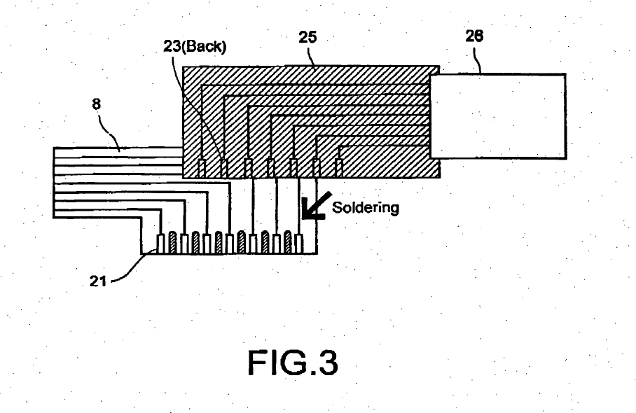

For avoiding the dielectric breakdown, an insulator may be provided between the adjacent two lands 21 as shown in FIG. 3, instead of keeping the predetermined interval. Any other possible way can be applied for avoiding the dielectric breakdown between the adjacent two lands 21.

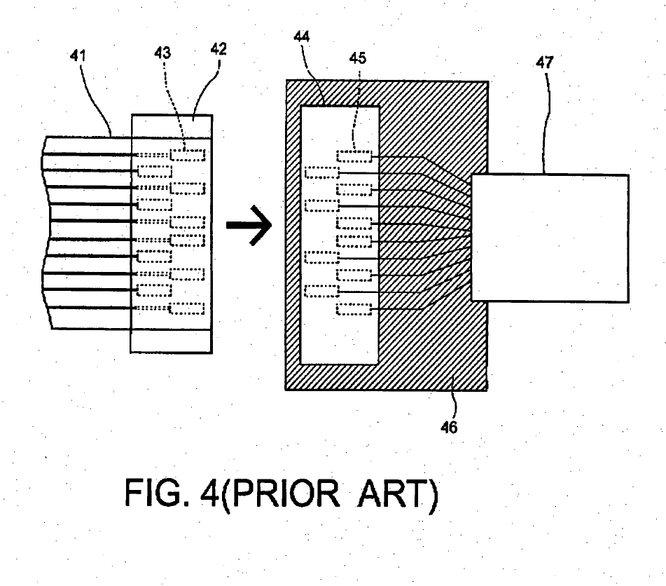

Compared to the above described land connection according to embodiments of the present invention, a conventional land connection will be described briefly with reference to FIG. 4. FIG. 4 is an illustration showing an exemplary configuration for explaining the conventional land connection according to a prior art of the present invention. Similar to FIG. 2A, a flexible printed wiring board 41 is connected to a connector 42 including a plurality of lands 43. Further, the connector 42 is connected to a connector 44 including a plurality of lands 45. The connector 44 provided in a printed wiring board 46 is also connected to a cable 47. The lands 43 provided in the connector 42 are connected to lands 45 provided in the connector 44. A typical feature of the lands 43 and 45 is a close-packed placement. In the connector 42, for example, adjacent two lands 43 are very close to each other. Consequently, if the predetermined high voltage is supplied to the lands 43, a dielectric breakdown is caused between the lands 43.

### First Embodiment

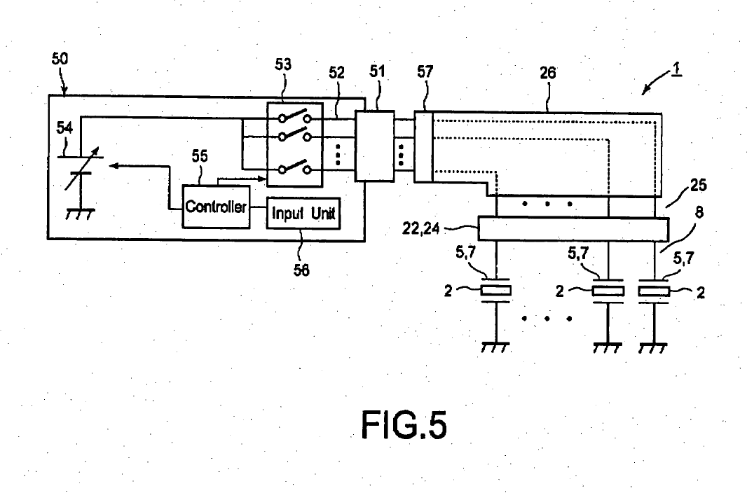

FIG. 5 is a block diagram showing a first exemplary configuration of a repolarization system according to a first embodiment of the present invention. As shown in FIG. 5, the repolarization system includes an ultrasonic probe repolarization apparatus 50 and the ultrasonic probe 1. The ultrasonic probe repolarization apparatus 50 includes a connector 51, connection lines 52, a switch unit 53, a high voltage power source 54, a controller 55, and an input unit 56. The connector 51 is detachably connected to a connector 57 of the ultrasonic probe 1. The connector 57 is connected to the cable 26. Other configuration of the ultrasonic probe 1 is similar to that described before. Therefore, explanations for the ultrasonic probe 1 are omitted herein.

The switch unit 53 includes a plurality of switching elements. The switching elements are switched on/off by the controller 55. At least a part of the switching elements corresponds to the lands 21 (23) one by one, respectively. In other words, all the switching elements may not always be used since the number of the lands 21 (23) depends on a type of the ultrasonic probe 1. Each of the corresponding switching elements is selected to switch on for supplying a predetermined high voltage to a corresponding land 21 (23). The switch unit 53 is connected to the connector 51 through the connection lines 52. The predetermined high voltage is generated by the high voltage power source 54. The controller 55 controls the switch unit 53 to switch on for a switch-on period of the switching elements. In addition, the controller 55 also controls the high voltage power source 54 so that the predetermined high voltage is generated. The predetermined high voltage varies depending on a type of the ultrasonic probe 1. The input unit 56 is used for inputting the predetermined voltage value and/or the switch-on period through an interface of the input unit 56. The controller 55 may further control the switch unit 53 and designate which switching elements to switch on (or which lands to be supplied with the predetermined high voltage). The controller 55 implements the above-described controls in accordance with the information input from the input unit 56.

The repolarization system configured as above will operate as follows.

An operator of the repolarization system inputs information of, for example, the predetermined high voltage and the switch-on period, from the input unit 56. The controller 55 receives the information and controls the high voltage power source 54 to generate the predetermined high voltage. The controller 55 also controls the switch unit 53 to switch on the switching elements for the input switch-on period.

The input unit 56 may alternatively include various buttons corresponding to various predetermined high voltages. Similarly, the input unit 56 may also include various buttons corresponding to various switch-on periods which may be appropriate for the repolarization. In such cases, the operator only needs to press one of the buttons appropriate for each case. The buttons may alternatively be provided as icons in a display window if provided in the ultrasonic probe repolarization apparatus 50. The operator may click one of the icons. Any of these inputs is construed as an instruction to the controller 55.

When the lands 21 (23) are supplied with the predetermined high voltage, the supplied voltage is applied to the electrodes 5 and 7. Since appropriate insulation is kept between one and the next of the lands 21 (23), the supplied high voltage is properly applied to the electrodes 5 and 7 for the switch-on period. Therefore, the transducers 2 are repolarized, which results in a recovery of the acoustic characteristic in the transducers 2. In response to the repolarization, the ultrasonic probe 1 can become reusable as an ultrasonic probe.

The predetermined high voltage maybe determined according to thickness of the transducers 2. The thickness of 1 [mm] may require a voltage 1 [kV]. For example, 2 millimeter-thick transducers may need to be supplied with a 2-kilovolt voltage. In addition, a voltage supply period (i.e., the switch-on period) may be approximately 30 to 60 seconds whatever the thickness is, for example.

### Second Embodiment

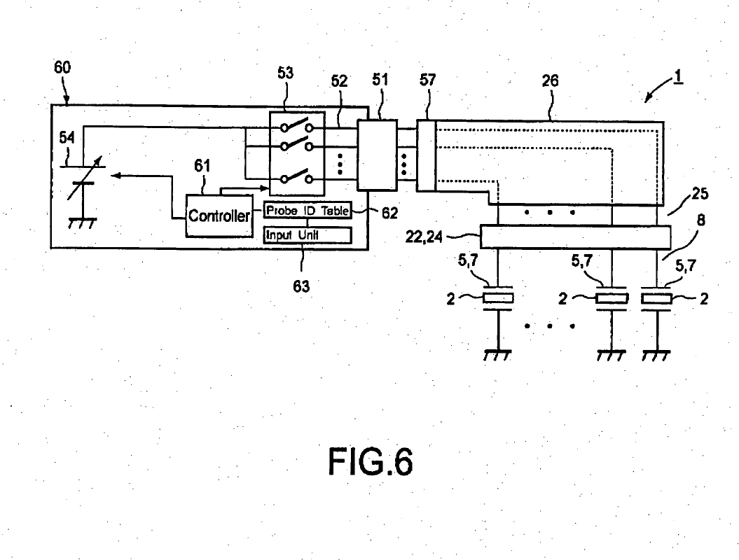

FIG. 6 is a block diagram showing a second exemplary configuration of the repolarization system according to a second embodiment of the present invention. In FIG. 6, components given the same reference numbers as those shown in FIGS. 1, 2A, and 5 will be operative in similar manners. Therefore, detailed explanations of such components are omitted herein.

In the repolarization system according to the second embodiment, an ultrasonic probe repolarization apparatus 60 includes a controller 61 instead of the controller 55, an ultrasonic probe identification table memory 62, and an input unit 63 instead of the input unit 56. The controller 61 controls the switch unit 53 and designates which switching elements to switch on (or which lands to be supplied with the predetermined high voltage). The controller 61 further controls the switch unit 53 to switch on for a switch-on period of the switching elements. In addition, the controller 61 also controls the high voltage power source 54 so that the predetermined high voltage is generated. The predetermined high voltage varies depending on a type of the ultrasonic probe 1. The input unit 63 is used for inputting information identifying a type of the ultrasonic probe 1 through an interface of the input unit 63.

The ultrasonic probe identification table memory 62 stores a first table showing a relationship between ultrasonic probe type identification information and supply voltage information. The supply voltage information shows various voltages high enough to polarize the electrodes 5 and 7. Which of the voltages to supply depends on a type of the ultrasonic probe 1. Further, the ultrasonic probe identification table memory 62 also stores a second table showing a relationship between ultrasonic probe type identification information and switching condition information. The switching condition information may include switch-on periods of the switch unit 53 and information of designating which switching elements to switch on. The switch-on period is determined to be long enough to polarize the electrodes 5 and 7. Which of the switch-on periods to apply depends on a type of the ultrasonic probe 1. It is necessary to determine the number of switching elements to switch on and/or to determine which switching elements to switch on since the number and relative positions of the lands 21 (23) are different among types of the ultrasonic probe 1. The first table and the second table may be combined to use as one table. The ultrasonic probe repolarization apparatus 60 may alternatively include only one of the first and second tables. In this case, the input according to the first embodiment may be combined in the input operation. Still further, the ultrasonic probe identification table memory 62 may be provided in the controller 61 as a part of features of the controller 61. In this case, the controller 61 refers to the ultrasonic probe identification table memory 62 for the controls.

The controller 61 implements the above-described controls in accordance with information supplied from the ultrasonic probe identification table memory 62 based on the information input by the input unit 63.

The repolarization system configured as above will operate as follows.

An operator of the repolarization system inputs information identifying a type of the ultrasonic probe 1 from the input unit 63. The ultrasonic probe identification table memory 62 receives the input information and compares the received information to the ultrasonic probe type identification information in the first table. As a result of the comparison, the supply voltage information corresponding to the ultrasonic probe type identification information identical to the received information is obtained and supplied to the controller 61. Similarly, the ultrasonic probe identification table memory 62 compares the received information to the ultrasonic probe type identification information in the second table. As a result of the comparison, the switching condition information corresponding to the ultrasonic probe type identification information identical to the received information is obtained and supplied to the controller 61. The controller 61 receives the supply voltage information and the switching condition information. The controller 61 controls the high voltage power source 54 to generate the predetermined high voltage based on the supply voltage information. Also based on the switching condition information, the controller 61 controls the switch unit 53 to switch on switching elements designated in the switching condition information for the switch-on period designated in the switching condition information. In case that the switch-on period is fixed for any case, the switch-on period is not required in the switching condition information.

The input unit 63 may alternatively include various buttons corresponding to various predetermined ultrasonic probe types. In such a case, the operator only needs to press one of the buttons appropriate for a type of the ultrasonic probe 1. The buttons may alternatively be provided as icons in a display window if provided in the ultrasonic probe repolarization apparatus 60. The operator may click one of the icons. Any of these inputs or information to be supplied from the ultrasonic probe identification table memory 62 is construed as an instruction to the controller 61.

Operations of the repolarization in the ultrasonic probe 1 are similar to those described in the first embodiment.

### Third Embodiment

When it is possible to categorize various types of the ultrasonic probe 1 into less number of types (or groups) in accordance with a supply voltage necessary for the repolarization, such categorized voltage types may be input to the controller of the ultrasonic probe repolarization apparatus so as to control the repolarization. This categorization is based on a fact that some types of ultrasonic probes can be repolarized with a similar voltage. Application of the controls according to such supply voltages may simplify operations by the operator and controls of the ultrasonic probe repolarization apparatus. One type of a supply voltage corresponds to one predetermined voltage to be supplied for the repolarization.

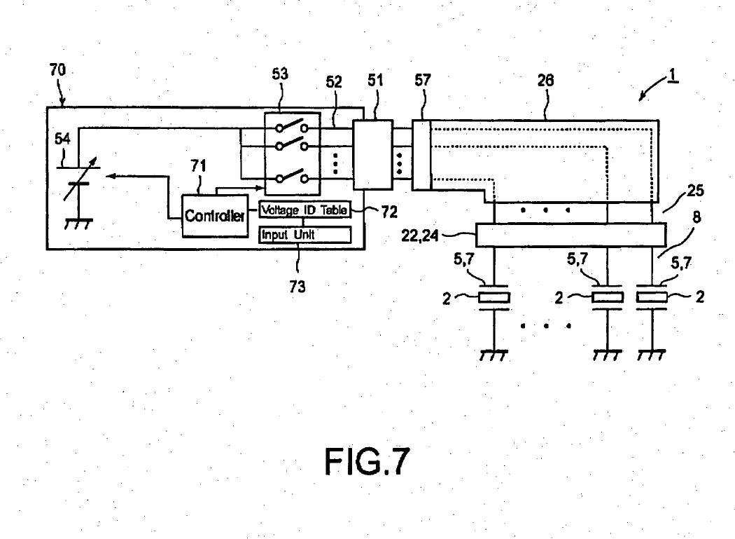

FIG. 7 is a block diagram showing a third exemplary configuration of the repolarization system according to a third embodiment of the present invention. In FIG. 7, components given the same reference numbers as those shown in FIGS. 1, 2A, and 5 will be operative in similar manners. Therefore, detailed explanations of such components are omitted herein.

In the repolarization system according to the third embodiment, an ultrasonic probe repolarization apparatus 70 includes a controller 71 instead of the controller 61, a supply voltage identification table memory 72, and an input unit 73 instead of the input unit 63. The controller 71 controls the switch unit 53 to switch on for a switch-on period of the switching elements. In addition, the controller 71 also controls the high voltage power source 54 so that the predetermined high voltage is generated. The predetermined high voltage varies depending on a type of the ultrasonic probe 1. The controller 71 may further control the switch unit 53 and designate which switching elements to switch on (or which lands to be supplied with the predetermined high voltage). The input unit 73 is used for inputting information identifying a supply voltage type of the predetermined high voltage for the ultrasonic probe 1 through an interface of the input unit 73.

The supply voltage identification table memory 72 stores a third table showing a relationship between supply voltage type identification information and supply voltage information. The supply voltage information shows various voltages high enough to polarize the electrodes 5 and 7. Which of the voltages to supply depends on a type of the ultrasonic probe 1. Further, the supply voltage identification table memory 72 also stores a fourth table showing a relationship between supply voltage type identification information and switching condition information. The switching condition information may include switch-on periods of the switch unit 53 and information of designating which switching elements to switch on. The switch-on period is determined to be long enough to polarize the electrodes 5 and 7. Which of the switch-on periods to apply depends on a type of the ultrasonic probe 1. It is necessary to determine the number of switching elements to switch on and/or to determine which switching elements to switch on since the number and relative positions of the lands 21 (23) are different among types of the ultrasonic probe 1. The third table and the fourth table may be combined to use as one table. The ultrasonic probe repolarization apparatus 70 may alternatively include only one of the third and fourth tables. In this case, the input according to the first embodiment may be combined in the input operation. Still further, the supply voltage identification table memory 72 may be provided in the controller 71 as a part of features of the controller 71. In this case, the controller 71 refers to the supply voltage identification table memory 72 for the controls.

The controller 71 implements the above-described controls in accordance with information supplied from the supply voltage identification table memory 72 based on the information input by the input unit 73.

The repolarization system configured as above will operate as follows.

An operator of the repolarization system inputs information identifying a supply voltage type of the predetermined high voltage for the ultrasonic probe 1 from the input unit 73. The supply voltage identification table memory 72 receives the input information and compares the received information to the supply voltage type identification information in the third table. As a result of the comparison, the supply voltage information corresponding to the supply voltage type identification information identical to the received information is obtained and supplied to the controller 71. Similarly, the supply voltage identification table memory 72 compares the received information to the supply voltage type identification information in the fourth table. As a result of the comparison, the switching condition information corresponding to the supply voltage type identification information identical to the received information is obtained and supplied to the controller 71. The controller 71 receives the supply voltage information and the switching condition information. The controller 71 controls the high voltage power source 54 to generate the predetermined high voltage based on the supply voltage information. Also based on the switching condition information, the controller 71 controls the switch unit 53 to switch on switching elements designated in the switching condition information for the switch-on period designated in the switching condition information. In case that the switch-on period is fixed for any case, the switch-on period is not required in the switching condition information.

The input unit 73 may alternatively include various buttons corresponding to various predetermined supply voltage types. In such a case, the operator only needs to press one of the buttons appropriate for a type of the supply voltage. The buttons may alternatively be provided as icons in a display window if provided in the ultrasonic probe repolarization apparatus 70. The operator may click one of the icons. Any of these inputs or information to be supplied from the supply voltage identification table memory 72 is construed as an instruction to the controller 71.

Operations of the repolarization in the ultrasonic probe 1 are similar to those described in the first embodiment.

### Fourth Embodiment

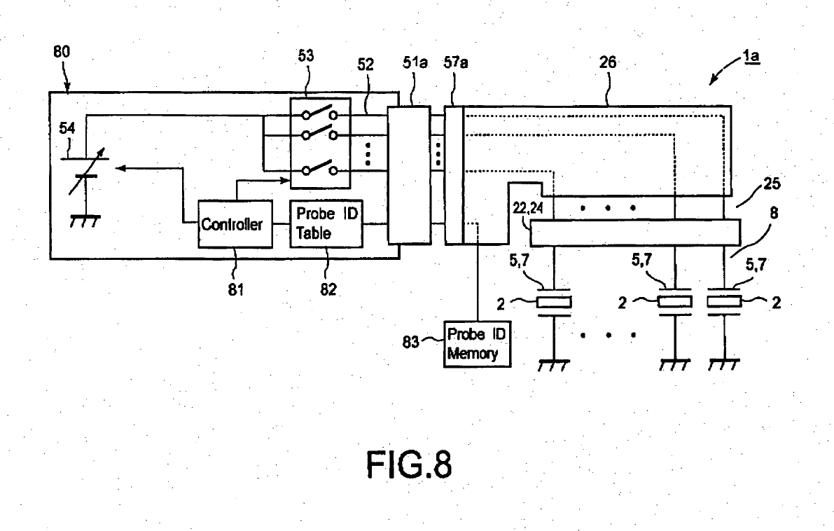

FIG. 8 is a block diagram showing a fourth exemplary configuration of the repolarization system according to a fourth embodiment of the present invention. In FIG. 8, components given the same reference numbers as those shown in FIGS. 1, 2A, and 5 will be operative in similar manners. Therefore, detailed explanations of such components are omitted herein.

In the repolarization system according to the fourth embodiment, an ultrasonic probe repolarization apparatus 80 includes a controller 81 instead of the controller 61, an ultrasonic probe identification table memory 82 instead of the ultrasonic probe identification table memory 62, and a connector 51a instead of the connector 51. An ultrasonic probe 1a includes a connector 57a instead of the connector 57, a cable 26a instead of the cable 26, and an ultrasonic probe identification memory 83.

The connector 57a is connected to the connector 51a. The connector 57a is also connected to the cable 26a. The cable 26a is similar to the cable 26, except for a connection to the ultrasonic probe identification memory 83. The ultrasonic probe identification memory 83 stores information identifying a type of the ultrasonic probe 1a. The information can be transferred to the ultrasonic probe identification table memory 82 through the cable 26a, the connector 57a, and the connector 51a.

In the ultrasonic probe repolarization apparatus 80, the controller 81 controls the switch unit 53 and designates which switching elements to switch on (or which lands to be supplied with the predetermined high voltage). The controller 81 further controls the switch unit 53 to switch on for a switch-on period of the switching elements. In addition, the controller 81 also controls the high voltage power source 54 so that the predetermined high voltage is generated. The predetermined high voltage varies depending on a type of the ultrasonic probe 1a.

The ultrasonic probe identification table memory 82 stores a fifth table showing a relationship between ultrasonic probe type identification information and supply voltage information. The supply voltage information shows various voltages high enough to polarize the electrodes 5 and 7. Which of the voltages to supply depends on a type of the ultrasonic probe 1a. Further, the ultrasonic probe identification table memory 82 also stores a sixth table showing a relationship between ultrasonic probe type identification information and switching condition information. The switching condition information may include switch-on periods of the switch unit 53 and information of designating which switching elements to switch on. The switch-on period is determined to be long enough to polarize the electrodes 5 and 7. Which of the switch-on periods to apply depends on a type of the ultrasonic probe 1a. It is necessary to determine the number of switching elements to switch on and/or to determine which switching elements to switch on since the number and relative positions of the lands 21 (23) are different among types of the ultrasonic probe la. The fifth table and the sixth table may be combined to use as one table. The ultrasonic probe repolarization apparatus 80 may alternatively include only one of the fifth and sixth tables. In this case, the input according to the first embodiment may be combined in the transferring operation as an input operation. Still further, the ultrasonic probe identification table memory 82 may be provided in the controller 81 as a part of features of the controller 81. In this case, the controller 81 refers to the ultrasonic probe identification table memory 82 for the controls.

The controller 81 implements the above-described controls in accordance with information supplied from the ultrasonic probe identification table memory 82 based on the information transferred from the ultrasonic probe identification memory 83 through the cable 26a, the connector 57a, and the connector 51a as interfaces.

According to the fourth embodiment of the present invention, the ultrasonic probe identification memory 83 may be provided in any part of the ultrasonic probe 1a.

The repolarization system configured as above will operate as follows.

An operator of the repolarization system connects the ultrasonic probe 1a to the ultrasonic probe repolarization apparatus 80 by putting the connector 57a into the connector 51a. In response to the connection or alternatively to a predetermined operation in the ultrasonic probe 1a and/or the ultrasonic probe repolarization apparatus 80, the information identifying a type of the ultrasonic probe 1a stored in the ultrasonic probe identification memory 83 is transferred to the ultrasonic probe identification table memory 82 through the cable 26a and the connectors 57a and 51a. The ultrasonic probe identification table memory 82 receives the transferred information and compares the received information to the ultrasonic probe type identification information in the first table. As a result of the comparison, the supply voltage information corresponding to the ultrasonic probe type identification information identical to the received information is obtained and supplied to the controller 81. Similarly, the ultrasonic probe identification table memory 82 compares the received information to the ultrasonic probe type identification information in the second table. As a result of the comparison, the switching condition information corresponding to the ultrasonic probe type identification information identical to the received information is obtained and supplied to the controller 81. The controller 81 receives the supply voltage information and the switching condition information. The controller 81 controls the high voltage power source 54 to generate the predetermined high voltage based on the supply voltage information. Also based on the switching condition information, the controller 81 controls the switch unit 53 to switch on switching elements designated in the switching condition information for the switch-on period designated in the switching condition information. In case that the switch-on period is fixed for any case, the switch-on period is not required in the switching condition information.

Operations of the repolarization in the ultrasonic probe 1a are similar to those described in the first embodiment.

### Fifth Embodiment

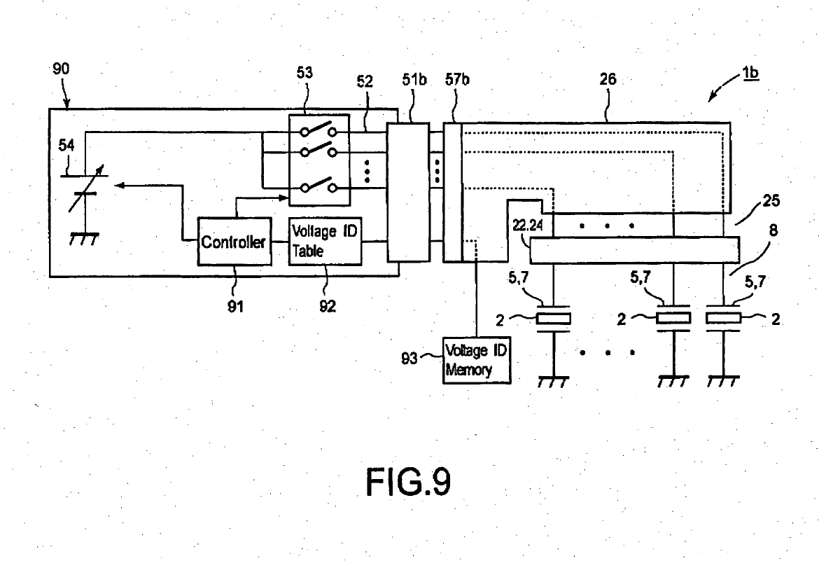

FIG. 9 is a block diagram showing a fifth exemplary configuration of the repolarization system according to a fifth embodiment of the present invention. In FIG. 9, components given the same reference numbers as those shown in FIGS. 1, 2A, and 5 will be operative in similar manners. Therefore, detailed explanations of such components are omitted herein.

In the repolarization system according to the fifth embodiment, an ultrasonic probe repolarization apparatus 90 includes a controller 91 instead of the controller 81, a supply voltage identification table memory 92 instead of the ultrasonic probe identification table memory 82, and a connector 51b instead of the connector 51a. An ultrasonic probe 1b includes a connector 57b instead of the connector 57a, a cable 26b instead of the cable 26a, and a supply voltage identification memory 93 instead of the ultrasonic probe identification memory 83.

The connector 57b is connected to the connector 51b. The connector 57b is also connected to the cable 26b. The cable 26b is similar to the cable 26a, except for a connection to the supply voltage identification memory 93. The supply voltage identification memory 93 stores information identifying a supply voltage type of the ultrasonic probe 1b. The information can be transferred to the supply voltage identification table memory 92 through the cable 26b, the connector 57b, and the connector 51b.

In the ultrasonic probe repolarization apparatus 90, the controller 91 controls the switch unit 53 and designates which switching elements to switch on (or which lands to be supplied with the predetermined high voltage). The controller 91 further controls the switch unit 53 to switch on for a switch-on period of the switching elements. In addition, the controller 91 also controls the high voltage power source 54 so that the predetermined high voltage is generated. The predetermined high voltage varies depending on a type of the ultrasonic probe 1b.

The supply voltage identification table memory 92 stores a seventh table showing a relationship between supply voltage type identification information and supply voltage information. The supply voltage information shows various voltages high enough to polarize the electrodes 5 and 7. Which of the voltages to supply depends on a type of the ultrasonic probe 1b. Further, the supply voltage identification table memory 92 also stores an eighth table showing a relationship between supply voltage type identification information and switching condition information. The switching condition information may include switch-on periods of the switch unit 53 and information of designating which switching elements to switch on. The switch-on period is determined to be long enough to polarize the electrodes 5 and 7. Which of the switch-on periods to apply depends on a type of the ultrasonic probe 1b. It is necessary to determine the number of switching elements to switch on and/or to determine which switching elements to switch on since the number and relative positions of the lands 21 (23) are different among types of the ultrasonic probe 1b. The seventh table and the eighth table may be combined to use as one table. The ultrasonic probe repolarization apparatus 90 may alternatively include only one of the seventh and eighth tables. In this case, the input according to the first embodiment may be combined in the transferring operation as an input operation. Still further, the supply voltage identification table memory 92 may be provided in the controller 91 as a part of features of the controller 91. In this case, the controller 91 refers to the supply voltage identification table memory 92 for the controls.

The controller 91 implements the above-described controls in accordance with information supplied from the supply voltage identification table memory 92 based on the information transferred from the supply voltage identification memory 93 through the cable 26b, the connector 57b, and the connector 51b as interfaces.

According to the fifth embodiment of the present invention, the supply voltage identification memory 93 may be provided in any part of the ultrasonic probe 1b.

The repolarization system configured as above will operate as follows.

An operator of the repolarization system connects the ultrasonic probe 1b to the ultrasonic probe repolarization apparatus 90 by putting the connector 57b into the connector 51b. In response to the connection or alternatively to a predetermined operation in the ultrasonic probe 1b and/or the ultrasonic probe repolarization apparatus 90, the information identifying a supply voltage type of the ultrasonic probe 1b stored in the supply voltage identification memory 93 is transferred to the supply voltage identification table memory 92 through the cable 26b and the connectors 57b and 51b. The supply voltage identification table memory 92 receives the transferred information and compares the received information to the supply voltage type identification information in the seventh table. As a result of the comparison, the supply voltage information corresponding to the supply voltage type identification information identical to the received information is obtained and supplied to the controller 91. Similarly, the supply voltage identification table memory 92 compares the received information to the supply voltage type identification information in the eighth table. As a result of the comparison, the switching condition information corresponding to the supply voltage type identification information identical to the received information is obtained and supplied to the controller 91. The controller 91 receives the supply voltage information and the switching condition information. The controller 91 controls the high voltage power source 54 to generate the predetermined high voltage based on the supply voltage information. Also based on the switching condition information, the controller 91 controls the switch unit 53 to switch on switching elements designated in the switching condition information for the switch-on period designated in the switching condition information. In case that the switch-on period is fixed for any case, the switch-on period is not required in the switching condition information.

Operations of the repolarization in the ultrasonic probe 1b are similar to those described in the first embodiment.

### Sixth Embodiment

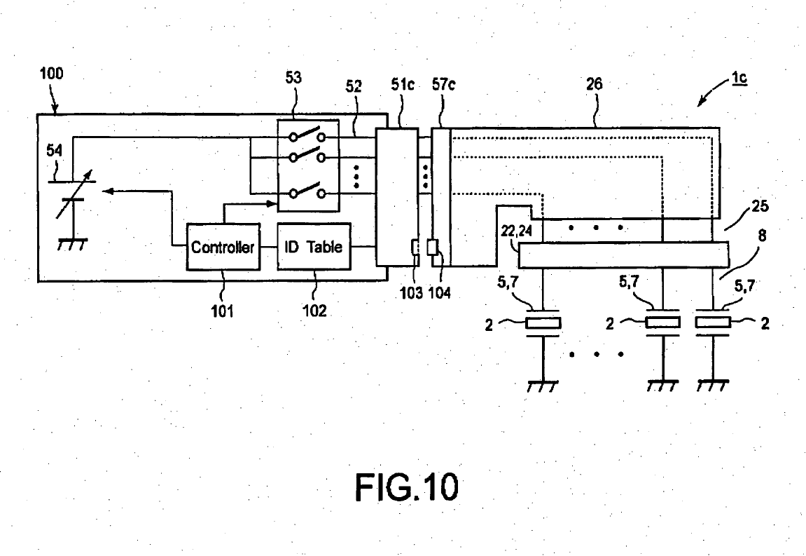

FIG. 10 is a block diagram showing a sixth exemplary configuration of the repolarization system according to a sixth embodiment of the present invention. In FIG. 10, components given the same reference numbers as those shown in FIGS. 1, 2A, and 5 will be operative in similar manners. Therefore, detailed explanations of such components are omitted herein.

In the repolarization system according to the sixth embodiment, an ultrasonic probe repolarization apparatus 100 includes a controller 101 instead of the controller 91, an identification table memory 102 instead of the supply voltage identification table memory 92, a first mechanism 103, and a connector 51c instead of the connector 51b. An ultrasonic probe 1c includes a connector 57c instead of the connector 57b and a second mechanism 104.

The connector 57c is connected to the connector 51c. The connector 57c is also connected to the cable 26. The second mechanism 104 is provided in the connector 57c. The second mechanism 104 is mechanically configured to have joint indicating predetermined information. The predetermined information is, for example, an ultrasonic probe type or a supply voltage type of the ultrasonic probe 1c. The second mechanism 104 is detachably attached to the first mechanism 103. The first mechanism 103 is provided in the connector 51c. The first mechanism is configured to have joint operative to respond to and recognize the first mechanism 104 and supply the identification table memory 102 with the predetermined information in accordance with the recognized mechanism. That is, electronic signals representing the predetermined information are generated in response to the operation of the first mechanism 103.

In the ultrasonic probe repolarization apparatus 100, the controller 101 controls the switch unit 53 and designates which switching elements to switch on (or which lands to be supplied with the predetermined high voltage). The controller 101 further controls the switch unit 53 to switch on for a switch-on period of the switching elements. In addition, the controller 101 also controls the high voltage power source 54 so that the predetermined high voltage is generated. The predetermined high voltage varies depending on a type of the ultrasonic probe 1c.

The identification table memory 102 stores a ninth table showing a relationship between identification information and supply voltage information. The supply voltage information shows voltages high enough to polarize the electrodes 5 and 7. Which of the voltages to supply depends on a type of the ultrasonic probe 1c. Further, the identification table memory 102 also stores a tenth table showing a relationship between identification information and switching condition information. The switching condition information may include switch-on periods of the switch unit 53 and information of designating which switching elements to switch on. The switch-on period is determined to be long enough to polarize the electrodes 5 and 7. Which of the switch-on periods to apply depends on a type of the ultrasonic probe 1c. It is necessary to determine the number of switching elements to switch on and/or to determine which switching elements to switch on since the number and relative positions of the lands 21 (23) are different among types of the ultrasonic probe 1c. The ninth table and the tenth table may be combined to use as one table. The ultrasonic probe repolarization apparatus 100 may alternatively include only one of the ninth and tenth tables. In this case, the input according to the first embodiment maybe combined in the attachment operation as an input operation. Still further, the identification table memory 102 may be provided in the controller 101 as a part of features of the controller 101. In this case, the controller 101 refers to the identification table memory 102 for the controls.

The controller 101 implements the above-described controls in accordance with information supplied from the identification table memory 102 based on the predetermined information recognized by the first mechanism 103 in response to the second mechanism 104.

The repolarization system configured as above will operate as follows.

An operator of the repolarization system connects the ultrasonic probe 1c to the ultrasonic probe repolarization apparatus 100 by putting the connector 57c into the connector 51c. In response to the connection, the second mechanism 104 is attached to the first mechanism 103. Accordingly, the predetermined information based on the second mechanism 104 is recognized by the first mechanism 103. Electronic signals representing the recognized predetermined information is supplied to the identification table memory 102. The identification table memory 102 receives the supplied information and compares the received information to the identification information in the ninth table. As a result of the comparison, the supply voltage information corresponding to the identification information identical to the received information is obtained and supplied to the controller 101. Similarly, the identification table memory 102 compares the received information to the identification information in the tenth table. As a result of the comparison, the switching condition information corresponding to the identification information identical to the received information is obtained and supplied to the controller 101. The controller 101 receives the supply voltage information and the switching condition information. The controller 101 controls the high voltage power source 54 to generate the predetermined high voltage based on the supply voltage information. Also based on the switching condition information, the controller 101 controls the switch unit 53 to switch on switching elements designated in the switching condition information for the switch-on period designated in the switching condition information. In case that the switch-on period is fixed for any case, the switch-on period is not required in the switching condition information.

Operations of the repolarization in the ultrasonic probe 1c are similar to those described in the first embodiment.

### Seventh Embodiment

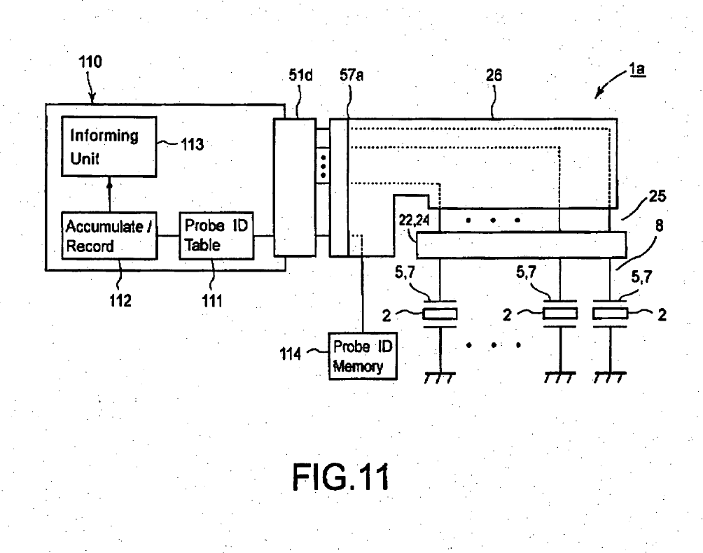

FIG. 11 is a block diagram showing a first exemplary configuration of an ultrasound imaging apparatus according to a seventh embodiment of the present invention. The ultrasound imaging apparatus includes a main body 110 and the ultrasonic probe 1a. Components of the ultrasonic probe 1a are similar to those of the ultrasonic probe 1a shown in FIG. 8. Therefore, explanations of such components will be omitted herein. The main body 110 includes an ultrasonic probe identification table memory 111, an accumulator 112, an informing unit 113, and a connector 51d. In addition, the main body 110 further includes a controller, a switch unit, a high voltage power source, and an input unit, which are not shown in FIG. 11. Needless to say, the main body 110 also includes components typically required for a conventional ultrasound imaging apparatus.

The ultrasonic probe 1a is detachably connected (or attached) to the main body 110 through the connectors 57a and 51d. The ultrasonic probe identification memory 114 stores information identifying the ultrasonic probe 1a. Information identifying the ultrasonic probe 1a stored in the ultrasonic probe identification memory 114 can be transferred to the ultrasonic probe identification table memory 111 through the cable 26a, the connector 57a, and the connector 51d.

In the main body 110, the controller controls the switch unit and designates which switching elements to switch on (or which lands to be supplied with a predetermined high voltage). The controller further controls the switch unit to switch on for a switch-on period of the switching elements. In addition, the controller also controls the high voltage power source so that the predetermined high voltage is generated. The predetermined high voltage varies depending on a type of the ultrasonic probe 1a.

The ultrasonic probe identification table memory 111 stores an eleventh table showing a relationship between ultrasonic probe identification information and supply voltage information. The supply voltage information shows various voltages high enough to polarize the electrodes 5 and 7. Which of the voltages to supply depends on a type of the ultrasonic probe 1a. Further, the ultrasonic probe identification table memory 111 also stores a twelfth table showing a relationship between ultrasonic probe identification information and switching condition information. The switching condition information may include switch-on periods of the switch unit and information of designating which switching elements to switch on. The switch-on period is determined to be long enough to polarize the electrodes 5 and 7. Which of the switch-on periods to apply depends on a type of the ultrasonic probe 1a. It is necessary to determine the number of switching elements to switch on and/or to determine which switching elements to switch on since the number and relative positions of the lands 21 (23) are different among types of the ultrasonic probe 1a. The eleventh table and the twelfth table may be combined to use as one table. The main body 110 may alternatively include only one of the eleventh and twelfth tables. In this case, inputs in a manner similar to the first embodiment may be combined in the transferring operation as an input operation. Still further, the ultrasonic probe identification table memory 111 may be provided in the controller as a part of features of the controller. In this case, the controller refers to the ultrasonic probe identification table memory 111 for the controls.

The accumulator 112 accumulates time during a use of the ultrasonic probe 1a. The use of the ultrasonic probe 1a may be defined, for example, that the ultrasonic probe 1a is supplied with a power for generating an ultrasound. Further, for example, the use of the ultrasonic probe 1a may indicate that the ultrasonic probe 1a generates an ultrasound. The accumulated time is recorded (or stored) in a recorder provided in the accumulator 112. The recorder may alternatively be provided independently or in the controller. The accumulation by the accumulator 112 is implemented every ultrasonic probe identification information. This is because the ultrasonic probe 1a may often be changed to others among a plurality of ultrasonic probes in accordance with a requirement in an ultrasound imaging.

The informing unit 113 gives a notice to an operator when the accumulated time has reached a predetermined time. The notice may be a suggestion that the ultrasonic probe 1a (i.e., the transducers 2) should be repolarized. The informing unit 113 may alternatively give a notice as a warning in a predetermined time before the time that the ultrasonic probe 1a should be repolarized. In this case, the notice may indicate when the ultrasonic probe 1a should be repolarized. Such indication may be displayed as a message in a display provided for displaying ultrasound images as a typical component of the ultrasound imaging apparatus. The ultrasound imaging apparatus may alternatively warn it in a sound, such as, for example, beep sound or a voice message. Instead of when to repolarize, a remaining time until when the ultrasonic probe 1a should be repolarized maybe calculated or estimated based on the accumulated time and be displayed in the display or warned in the sound.

Usually the predetermined time is determined based on an image quality of images displayed in the display. If the depolarization progresses to a certain extent, the image quality is deteriorated to become beyond the permissible quality range. The predetermined time may be a time that such deterioration is caused and may depend on a type of the ultrasonic probe 1a or the transducers 2. Typically, the predetermined time may be about one year.

When the operator inputs an instruction through the input unit in response to the notice given by the informing unit 113, the controller controls the switch unit and the high voltage power source so that the repolarization is implemented as described before.

### Eighth Embodiment

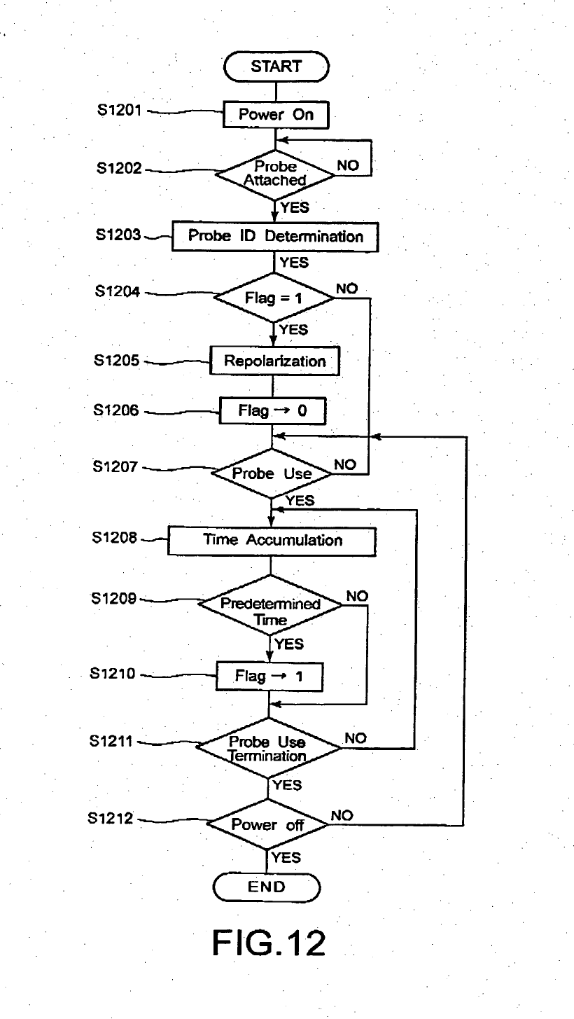

FIG. 12 is a flowchart showing an example of operations of an ultrasound imaging apparatus according to an eighth embodiment of the present invention. The ultrasound imaging apparatus according to the eighth embodiment will be described with reference to FIGS. 11 and 12. The ultrasound imaging apparatus is, however, not necessarily required to incorporate the informing unit 113 according to the eighth embodiment.

The ultrasound imaging apparatus automatically implements the repolarization under a predetermined condition as shown in FIG. 12. Operations shown in steps of FIG. 12 are implemented by the controller, the ultrasonic probe identification table memory 111, the accumulator 112, and so on.

When the ultrasound imaging apparatus is switched on for operations (step S1201), it is determined whether the ultrasonic probe 1a is attached to the main body 110 or not (step S1202). In response to the determination of the attachment, ultrasonic probe identification information of the ultrasonic probe 1a is transferred from the ultrasonic probe 1a to the main body 110 and determined (or recognized) (step S1203). After the determination, a flag corresponding to the determined ultrasonic probe identification information is determined whether it is one or zero (step S1204). If the flag is one, the main body 110 of the ultrasound imaging apparatus automatically implements the repolarization on the ultrasonic probe 1a (or the transducers 2) (step S1205) The flag is changed to zero (step S1206).

After step S1206 and when the flag is not one in step S1204, it is determined whether the ultrasonic probe 1a is being used or not (step S1207). In the determination of the use of the ultrasonic probe 1a, time accumulation is implemented (step S1208). The accumulated time is recorded in the recorder. When the accumulated time has reached a predetermined time (step S1209), the flag is changed to one (step S1210). The predetermined time may be set, taking into consideration, for example, average time estimated to be required for one day use of the ultrasonic probe 1a as a margin. The time accumulation continues during the use of the ultrasonic probe 1a. When the use of the ultrasonic probe 1a is terminated (step S1211), the time accumulation is terminated. Further, in response to switching off the power supply to the ultrasound imaging apparatus, the operations are terminated (step S1212).

According to the operations described above, when the ultrasonic probe 1a is used and accumulated time has reached the predetermined time, the repolarization is automatically implemented at the beginning of the next use of the ultrasonic probe 1a. Therefore, the operator needs neither to pay attention to the time for the repolarization nor to perform a predetermined operation for the repolarization.

As a first alternative example, when it is determined that the accumulated time has reached a predetermined time in step S1209, the repolarization may be automatically implemented in response to the termination of the use of the ultrasonic probe 1a in step S1211.

Still further, it can be a second alternative example that an operator's confirmation is requested by displaying such a confirmation request message in the display or by a voice message in advance of implementing the repolarization in step S1205 or in the above first alternative example.

### Ninth Embodiment

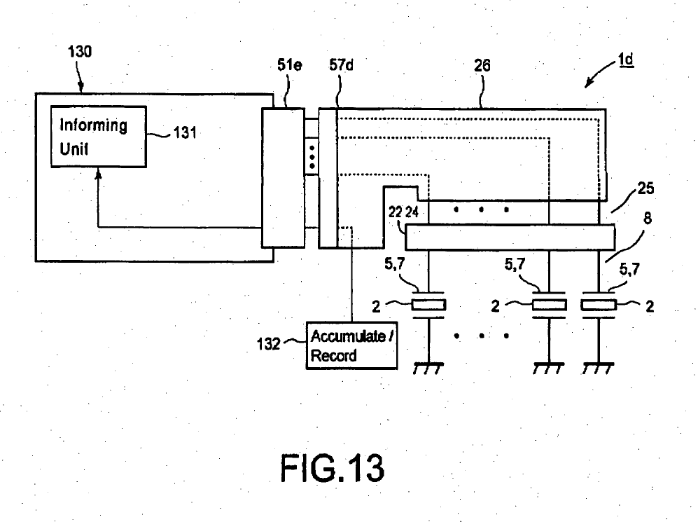

FIG. 13 is a block diagram showing a second exemplary configuration of the ultrasound imaging apparatus according to a ninth embodiment of the present invention. In FIG. 13, components given the same reference numbers as those shown in FIGS. 1 and 2A, and 5 will be operative in similar manners. Therefore, detailed explanations of such components are omitted herein.

The ultrasound imaging apparatus includes a main body 130 and an ultrasonic probe 1d. The main body 130 includes an informing unit 131 and a connector 51e. In addition, the main body 130 further includes a controller, a switch unit, a high voltage power source, an ultrasonic probe identification table memory, and an input unit, which are not shown in FIG. 13. Needless to say, the main body 130 also includes components typically required for a conventional ultrasound imaging apparatus. The ultrasonic probe 1d includes a connector 57d, a cable 26d, and an accumulator 132, in addition to the components shown in FIGS. 1 and 2A.

The connector 57d is connected to the connector 51e. The connector 57d is also connected to the cable 26d. The cable 26d is similar to the cable 26, except for a connection to the accumulator 132. Therefore, the ultrasonic probe 1d is detachably connected (attached) to the main body 130 through the connectors 57d and 51e. The accumulator 132 accumulates time during a use of the ultrasonic probe 1d. The accumulated time is recorded (or stored) in a recorder provided in the accumulator 132. The recorder may alternatively be provided independently in the ultrasonic probe 1d or in the main body 130.

The accumulated time is transferred to the controller provided in the main body 130. The controller determines whether the accumulated time has reached a predetermined time. Alternatively, when it is determined in the accumulator 132 that the accumulated time has reached the predetermined time, instruction signals may be sent to the informing unit 130 through the cable 26d, the connector 57d, and the connector 51e.

In the main body 130, the controller controls the switch unit and designates which switching elements to switch on (or which lands to be supplied with a predetermined high voltage). The controller further controls the switch unit to switch on for a switch-on period of the switching elements. In addition, the controller also controls the high voltage power source so that the predetermined high voltage is generated. The predetermined high voltage varies depending on a type of the ultrasonic probe 1d.

The ultrasonic probe identification table memory stores a table showing a relationship between ultrasonic probe identification information and supply voltage information. The supply voltage information shows various voltages high enough to polarize the electrodes 5 and 7. Which of the voltages to supply depends on a type of the ultrasonic probe 1d. Further, the ultrasonic probe identification table memory also stores another table showing a relationship between ultrasonic probe identification information and switching condition information. The switching condition information may include switch-on periods of the switch unit and information of designating which switching elements to switch on. The switch-on period is determined to be long enough to polarize the electrodes 5 and 7. Which of the switch-on periods to apply depends on a type of the ultrasonic probe 1d. It is necessary to determine the number of switching elements to switch on and/or to determine which switching elements to switch on since the number and relative positions of the lands 21 (23) are different among types of the ultrasonic probe id. The table and another table may be combined to use as one table. The main body 130 may alternatively include only one of the table and another table. In this case, inputs in a manner similar to the first embodiment may be combined as an input operation. Still further, the ultrasonic probe identification table memory may be provided in the controller as a part of features of the controller. In this case, the controller refers to the ultrasonic probe identification table memory for the controls.

The informing unit 130 gives a notice to an operator when the accumulated time has reached the predetermined time. As described above, the informing unit 130 is operative in response to the instruction signals from the accumulator 132 or the controls by the controller. The notice may be a suggestion that the ultrasonic probe id (i.e., the transducers 2) should be repolarized. The informing unit 130 may alternatively give a notice as a warning in a predetermined time before the time that the ultrasonic probe id should be repolarized. In this case, the notice may indicate when the ultrasonic probe id should be repolarized. Such indication may be displayed as a message in a display provided for displaying ultrasound images as a typical component of the ultrasound imaging apparatus. The ultrasound imaging apparatus may alternatively warn it in a sound, such as, for example, beep sound or a voice message. Instead of when to repolarize, a remaining time until when the ultrasonic probe 1d should be repolarized may be calculated and estimated based on the accumulated time and be displayed in the display or warned in the sound.

When the operator inputs an instruction through the input unit in response to the notice given by the informing unit 131, the controller controls the switch unit and the high voltage power source so that the repolarization is implemented as described before.

Although the ultrasound imaging apparatus has been described only in the seventh to the ninth embodiments, any idea of the embodiments described for the repolarization system (i.e., the first to the sixth embodiments) can also be applied to the ultrasound imaging apparatus.

The embodiments of the present invention described above are examples described only for making it easier to understand the present invention, and are not described for the limitation of the present invention. Consequently, each component and element disclosed in the embodiments of the present invention may be redesigned or modified to its equivalent within a scope of the present invention. Furthermore, any possible combination of such components and elements may be included in a scope of the present invention as long as an advantage similar to those obtained according to the above disclosure in the embodiments of the present invention is obtained.

Numerous modifications and variations of the present invention are possible in light of the above teachings. It is therefore to be understood that within the scope of the appended claims, the invention may be practiced otherwise than as specifically described herein.

1. A repolarization system including an ultrasonic probe, the system comprising:
a plurality of transducers provided in the ultrasonic probe, each one or more of the transducers being provided with a pair of electrodes;
a voltage generator configured to generate a predetermined voltage high enough to repolarize the transducers;
a switch configured to switch to supply the voltage to the pair of electrodes;
a connector configured to connect the pair of electrodes to the switch through a plurality of lands each of which corresponding to the pair of electrodes, the lands being provided to keep insulation enough to repolarize the transducers among the lands;
an interface configured to give an instruction; and
a controller configured to control the voltage generator to generate the predetermined voltage and to control the switch to supply the predetermined voltage to the pair of electrodes in accordance with the instruction.

2. The system according to claim 1, wherein the ultrasonic probe is detachable from the system.

3. The system according to claim 1, wherein the system is incorporated in an ultrasound imaging apparatus.

4. The system according to claim 1, wherein the lands are provided at predetermined intervals so as to keep the insulation between each two of the lands.

5. The system according to claim 1, wherein there is an insulator between each two of the lands.

6. The system according to claim 1, further comprising a memory configured to store a table showing a relationship between ultrasonic probe type identification information and supply voltage information; and an input unit configured to input information identifying a type of the ultrasonic probe as the instruction, and wherein the controller determines the predetermined voltage based on the supply voltage information corresponding to the ultrasonic probe type identification information identical to the information input by the input unit.

7. The system according to claim 1, further comprising a memory configured to store a table showing a relationship between ultrasonic probe type identification information and switching condition information; and an input unit configured to input information identifying a type of the ultrasonic probe as the instruction, and wherein the controller controls the switch based on the switching condition information corresponding to the ultrasonic probe type identification information identical to the information input by the input unit.

8. The system according to claim 1, further comprising a memory configured to store a table showing a relationship between supply voltage type identification information and supply voltage information; and an input unit configured to input information identifying a supply voltage type of the ultrasonic probe as the instruction, and wherein the controller determines the predetermined voltage based on the supply voltage information corresponding to the supply voltage type identification information identical to the information input by the input unit.

9. The system according to claim 1, further comprising an input unit configured to input a value of the predetermined voltage as the instruction.

10. The system according to claim 1, further comprising an input unit configured to input a time period for which the predetermined voltage is supplied to the pair of the electrodes.

11. The system according to claim 1, further comprising a memory configured to store a table showing a relationship between ultrasonic probe type identification information and supply voltage information, wherein the ultrasonic probe is further configured to store information identifying a type of the ultrasonic probe, and wherein the controller determines the predetermined voltage based on the supply voltage information corresponding to the ultrasonic probe type identification information identical to the information provided from the ultrasonic probe as the instruction.

12. The system according to claim 1, further comprising a memory configured to store a table showing a relationship between ultrasonic probe type identification information and switching condition information, wherein the ultrasonic probe is further configured to store information identifying a type of the ultrasonic probe, and wherein the controller controls the switch based on the switching condition information corresponding to the ultrasonic probe type identification information identical to the information provided from the ultrasonic probe as the instruction.

13. The system according to claim 1, further comprising a memory configured to store a table showing a relationship between supply voltage type identification information and supply voltage information, wherein the ultrasonic probe is further configured to store information identifying a supply voltage type of the ultrasonic probe, and wherein the controller determines the predetermined voltage based on the supply voltage information corresponding to the supply voltage type identification information identical to the information provided from the ultrasonic probe as the instruction.

14. The system according to claim 1, further comprising a first mechanism configured to respond to a second mechanism provided in the ultrasonic probe so as to supply a predetermined information based on the second mechanism as the instruction; and a memory configured to store a table showing a relationship between the predetermined information and supply voltage information, wherein the controller determines the predetermined voltage based on the supply voltage information corresponding to the predetermined information supplied through the first mechanism.

15. The system according to claim 1, wherein the switch includes a plurality of switching elements and wherein the controller controls one or more of the switching elements to connect the voltage generator to the pair of electrodes provided for each of one or more of the transducers, the one or more of the transducers corresponding to the one or more of the switching elements.

16. The apparatus according to claim 1, wherein the controller is further configured to vary time to control the switch to supply the predetermined voltage in accordance with the instruction.

17. An ultrasound imaging apparatus for obtaining an ultrasound image through an ultrasonic probe, the apparatus comprising:
a plurality of transducers provided in the ultrasonic probe and configured to generate an ultrasound so as to obtain the ultrasound image, each one or more of the transducers being provided with a pair of electrodes;
a voltage generator configured to generate a predetermined voltage high enough to repolarize the transducers;
a switch configured to switch to supply the voltage to the pair of electrodes;
a connector configured to connect the pair of electrodes to the switch through a plurality of lands each of which corresponding to the pair of electrodes, the lands being provided to keep insulation enough to repolarize the transducers among the lands;
an interface configured to give an instruction; and
a controller configured to control the voltage generator to generate the predetermined voltage and to control the switch to supply the predetermined voltage to the pair of electrodes in accordance with the instruction.

18. The apparatus according to claim 17, further comprising an accumulator configured to accumulate a time when the transducers generate the ultrasound; a recorder configured to record the accumulated time; and a notice component configured to give a notice according to the accumulated time.

19. The apparatus according to claim 18, wherein the notice component gives the notice in response that the accumulated time has reached a predetermined time.

20. The apparatus according to claim 19, wherein the notice component gives the notice to suggest repolarizing the transducers.

21. The apparatus according to claim 18, wherein the notice component gives the notice to suggest when to repolarize the transducers.

22. The apparatus according to claim 18, wherein the notice component includes a display configured to display a remaining time estimated based on the accumulated time until repolarizing the transducers.

23. The apparatus according to claim 17, further comprising an accumulator configured to accumulate a time when the transducers generate the ultrasound; and a recorder configured to record the accumulated time, and wherein the controller is further configured to determine a power supply for the apparatus; and wherein the controller automatically controls the switch to supply the predetermined voltage to the pair of electrodes in response to determining the power supply when the accumulated time has reached a predetermined time at a time of a last termination of the power supply.

24. The apparatus according to claim 17, further comprising an accumulator configured to accumulate a time when the transducers generate the ultrasound; and a recorder configured to record the accumulated time, and wherein the controller is further configured to determine that the transducers terminate the ultrasound; and wherein the controller automatically controls the switch to supply the predetermined voltage to the pair of electrodes in response to determining the ultrasound termination when the accumulated time has reached a predetermined time at a time of the ultrasound termination.

25. The apparatus according to claim 18, wherein the accumulator and the recorder are provided in the ultrasonic probe.

26. The apparatus according to claim 17, wherein the ultrasonic probe is detachable from the apparatus.

27. An ultrasonic probe which is connectable to a voltage supply apparatus, the probe comprising:
a plurality of transducers, each one or more of which being provided with a pair of electrodes;
a plurality of lands each of which configured to correspond to thee pair of electrodes and provided to keep insulation enough to repolarize the transducers among the lands;
a cable configured to include a plurality of voltage supply lines connected to the lands, respectively; and
a connector configured to connect the cable to the voltage supply apparatus, wherein a predetermined voltage is supplied to the transducers through the lands.
